(19) United States
(12) Patent Application Publication
(10) Pub. No.: US 2026/0198927 A1
(43) Pub. Date: Jul. 16, 2026

(54) SINGLE-HANDED LIGATION DEVICE

(71) Applicant: The Research Foundation for the State University of New York, Albany, NY (US)

(72) Inventors: Zachary Plona, Stony Brook, NY (US); Aurora Pryor, Stony Brook, NY (US); William Beasley, Stony Brook, NY (US)

(21) Appl. No.: 19/136,396

(22) PCT Filed: Dec. 7, 2023
(86) PCT No.: PCT/US2023/082900

§ 371 Date: Jun. 6, 2025

Related U.S. Application Data
(60) Provisional application No. 63/431,243, filed on Dec. 8, 2022.

(51) Int. Cl. A61B 17/12; A61B 17/00

(52) U.S. Cl. CPC A61B 17/12013; A61B 2017/0023; A61B 2017/00367; A61B 2017/00424

(57) ABSTRACT

The disclosure is directed to an efficient single-handed ligation device comprising a variety of mechanisms incorporated with a surgical laparoscopic cannula for use in tightening a suture knot within the body.

### BACKGROUND OF THE INVENTION

This Application claims priority to U.S. Provisional Application No. 63/431,243, filed Dec. 8, 2022. The content of this earlier filed application is hereby incorporated by reference herein in its entirety.

The instant invention relates generally to the field of surgical instruments and methods. More particularly, the present invention relates to an apparatus and method for laparoscopic surgery using a single-handed suture ligation device.

Laparoscopy is a popular technique of minimally invasive surgery because of Its significant advantages over conventional surgical techniques: reduced surgical trauma and scarring, substantially less pain and discomfort, significantly decreased risks of infection, and shortened recuperation times, as well as shorter, less expensive hospital stays.

In laparoscopy, all of the steps of a surgical procedure, for example, dissection, cutting, suturing, and ligating are performed by means of elongated instruments that are inserted into the abdominal cavity via trocars that, in tum, are inserted through small, “minimally invasive” incisions or punctures.

One of the more difficult surgical skills to acquire is that of laparoscopic knot-tying or ligation of vessels and tissue pedicles and the like. Of the various laparoscopic ligation methods, such as endostaplers, intracorporeal knots, endoclips and the like, surgeons frequently use ligation loops for purposes of ligation. Laparoscopic procedures utilizing suture or endoloops include: appendectomy, for ligation of the appendix, or for retraction of the appendix; cholecystectomy, for ligation of the cystic duct in a top-down approach, or for closure of hole in the gallbladder; wedge resection of gastric smooth muscle tumor, wherein the endoloop can be placed around the external projection of the tumor for retraction; mid-esophageal diverticulum resection, for use of retraction of esophagus and tying of penrose drain; right hemicolectomy, to control bleeding at the ileocolic pedicle; total abdominal colectomy, to control bleeding at the ileocolic pedicle; pediatric inguinal hernia repair, for internal ligation of the hernia sac; sleeve gastrectomy, for help with specimen retraction; and the utilization of an endoloop in surgery for ectopic pregnancy. Additionally, endoloops are sometimes used in an unplanned manner to control bleeding during a surgical procedure.

While there have been several suture-tying or ligation devices developed in the prior art, none of these are particularly well-suited for single-handed use in laparoscopic procedures.

For example, U.S. Pat. No. 8,021,376, to Takemoto et al. discloses an endoscopic suturing device that includes a needle mounted on at least one of a first and second actuating member with insertion components. The '376 patent discloses a suturing device, which uses a curved needle to drive suture into tissue through an externally operated trigger.

U.S. Pat. No. 8,157,824, to Kimura et al. discloses a clip like device having arms which grip tissue, a press tube serving as a clamping member which is fitted and mounted on the clip to close the arms of the clip, a coupling member which can be inserted into the press tube and engages with the clip, and projections serving as lock portions which hold the arms of the clip in a closed state when the clip engages with the press tube.

U.S. Pat. No. 9,023,066, to Pasricha et al. discloses a suturing-ligating device having a distal end portion and a proximal end portion, that carries at least one of suturing and ligation member. The '066 patent discloses a system of suture ligation which utilizes a movable stopper element. This element can be moved along the suture and tightened at different locations with friction. While this device can be used to ligate tissue with a loop of suture the loop is secured with a stopper as opposed to a pre-tied suture knot. Further, the '066 discloses that the suturing-ligating device is designed to easily release tissue from a sutured or ligated state by a simple operation.

There are various commercial ligating loop suture devices, for example, the manufacturer Covidien produces a ligating loop with suture that consists of a narrow nylon carrier through which a threaded 53 cm length absorbable suture extends from the distal end of the carrier in the form of a loop, having a sliding knot which secures itself when tightened. A delivery system is used for the introduction of the loop and carrier into an appropriately sized trocar sleeve in a laparoscopic procedure. The Covidien product requires the use of two hands, one to hold the suture carrier and the other to pull the suture back from the distal end to retract the suture and tighten the ligating loop.

Johnson & Johnson Surgical Technologies manufactures an endoloop ligature consisting of an 18-inch-long ligature in a plastic tube that is narrow at one end and scored at the other, where it is attached. The suture is formed in a ligature loop with a knot, and once the ligature is in place, a second hand is needed to snap the scored end and pull upward to tighten the loop and secure the knot.

The Olympus company provides a single-use poly-loop ligation device that is preassembled and can be used for endoscopic ligation of polyps. The loop design snares and securely ligates small and large polyps, minimizing the risk of sudden bleeding during polypectomy. Similar to other ligation devices, this device requires the use of two hands and/or the surgeon to provide a motion away from the device to secure the ligation.

Thus, there has been an as-yet unfulfilled need for a ligature tying device that is suitable for use through a trocar for laparoscopic procedures, that includes self-contained means for manipulating and tightening the ligature knot through the trocar with the use of only one hand. Furthermore, it would be advantageous to provide such a device that is also simple to manufacture and to use, and that does not require the use of new, complex suture knots. It would be further advantageous to provide a device of this nature that can be made in a disposable or single use embodiment.

### BRIEF SUMMARY OF THE INVENTION

The embodiments disclosed herein are directed to a single-handed ligation device for tying a suture around a selected body tissue. In embodiments, the device contains a suture having a proximate end and a distal end. The distal end of the suture has a suture loop and a suture knot, both of which are external to the device. In other embodiments disclosed herein, the device has an elongated cannula that extends axially between a proximate end and a distal end. According to an embodiment, the distal end of the cannula further has a tapered end that provides a distal orifice. According to another embodiment, the distal orifice is sized to provide passage for the suture and minimize fluid and gas entry into the device from the patient's abdomen. The distal orifice is small enough to prevent the suture knot from entering the cannula and can be used to manipulate and move the suture knot into position during the ligation procedure. According to other embodiments, the device also has a cap that has an open end that is affixed to the proximate end of the cannula. The cap also has a closed end that has a slot and a plurality of cap fulcrum holes. The cap also provides a flange that encircles the cannula. The device also contains a plunger with a distal end that is opposite the plunger's proximate end. The proximate end of the plunger has a knob or cap for actuating the plunger. The distal end of the plunger is sized for axial movement through the slot and proximate end of the cannula. The plunger also has a plurality of plunger fulcrum holes at its distal end.

According to embodiments disclosed herein, the single-handed ligation provides a single suture leverage when the plunger is depressed, and suture is configured to ascend from distal end of the cannula where the proximate end of the suture passes through a first cap fulcrum hole and then reverses direction down a second cap fulcrum hole or the slot where the proximate end of suture is then tethered to a first plunger fulcrum hole at the distal end of the plunger.

According to embodiments disclosed herein, the single-handed ligation device provides a double suture leverage when the plunger is depressed, and suture is configured to ascend from distal end of the cannula where the proximate end of the suture passes through a first cap fulcrum hole and then reverses direction down a second cap fulcrum hole or the slot and further reverses direction passing through a first plunger fulcrum hole at the distal end of the plunger where the proximate end of suture is then tethered to a third cap fulcrum hole.

According to other embodiments disclosed herein, the single-handed ligation device provides a triple suture leverage when the plunger is depressed, and suture is configured to ascend from distal end of the cannula where the proximate end of the suture passes through a first cap fulcrum hole and then reverses direction down a second cap fulcrum hole or the slot and further reverses direction passing through a first plunger fulcrum hole at the distal end of the plunger and then the proximate end of suture passes through a third cap fulcrum hole and further reverses direction down a fourth cap fulcrum hole where the proximate end of suture is then tethered to a second plunger fulcrum hole.

According to other embodiments disclosed herein, the single suture leverage configuration provides a 1:1 ratio of the distance traveled by the plunger is equal to the length of suture retracted by the device. According to other embodiments disclosed herein, the double suture leverage configuration provides a 1:2 ratio of the distance traveled by the plunger is equal to one-half the length of suture retracted by the device. According to other embodiments disclosed herein, the triple suture leverage configuration provides a 1:3 ratio of the distance traveled by the plunger is equal to one-third the length of suture retracted by the device.

According to other embodiments disclosed herein, the single-handed ligation device's cap has a T shaped slot, or a cross shaped slot, or round shaped slot and the plunger's distal end is appropriately configured and sized for axial movement through the slot and proximate end of the cannula. According to other embodiments disclosed herein, the cap and cannula are manufactured as one piece. According to other embodiments disclosed herein, the device contains a cutting device for cutting the suture after deployment. According to other embodiments disclosed herein, the cutting device for cutting the suture is positioned on the distal end of the plunger and/or integrated as part of the cannula. According to other embodiments disclosed herein, the device is made of a plastic material and is disposable. According to other embodiments disclosed herein, the device is made of a metal material and can be sterilized. According to other embodiments disclosed herein, the plunger can be removed after the suture knot has been deployed. According to other embodiments disclosed herein, the proximate end of the cannula accommodates a mean for retraction of the suture into the device. According to other embodiments disclosed herein, the proximate end of the cannula accommodates a rotational round plunger, or a finger wheel tensioner, or a finger bar tensioner, or a finger rachet tensioner, or a finger-trigger rachet tensioner, or finger clutch tensioner, for retraction of the suture into the device. According to other embodiments disclosed herein, the device of claim 1, wherein the cannula is a hinged cannula that when moved provides retraction of the suture into the device.

The single-handed ligation device can be manufactured so that the cannula and cap form one continuous piece, i.e., a unibody, to accommodate the suture and the plunger. The uniform cannula and cap provide a flange or accommodation at the proximate end to aid a user in the plunging action required to actuate the device. Accordingly, the unibody single-handed ligation device is preloaded with suture, a suture loop, and a pre-tied suture knot, so that the plunger and suture loop are positioned for immediate actuation and deployment, as needed.

The suture can be configured within the device to provide suture leverage, i.e., mechanical leverage over the length of suture that can be retracted when actuating the device's plunger, as more fully explained herein below.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is an illustrative view presenting a configuration of a single-handed ligation device according to an embodiment of the present invention.

FIG. 1B is an illustrative perspective view of the proximate end of the single-handed ligation device according to an embodiment of the present invention.

FIG. 1C is an illustrative view of the single-handed ligation device utilizing an internal path for the suture according to an embodiment of the present invention.

FIG. 2A is a perspective top view of an internal cap according to an embodiment of the present invention.

FIG. 28 is a perspective side view of an internal cap and an external suture configuration of the single-handed ligation device according to an embodiment of the present invention.

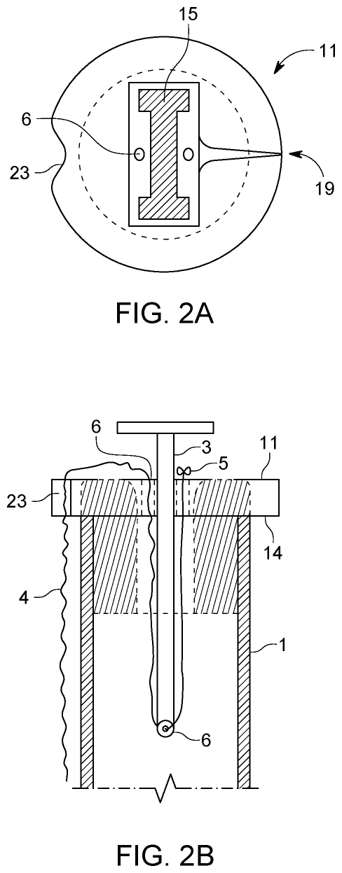

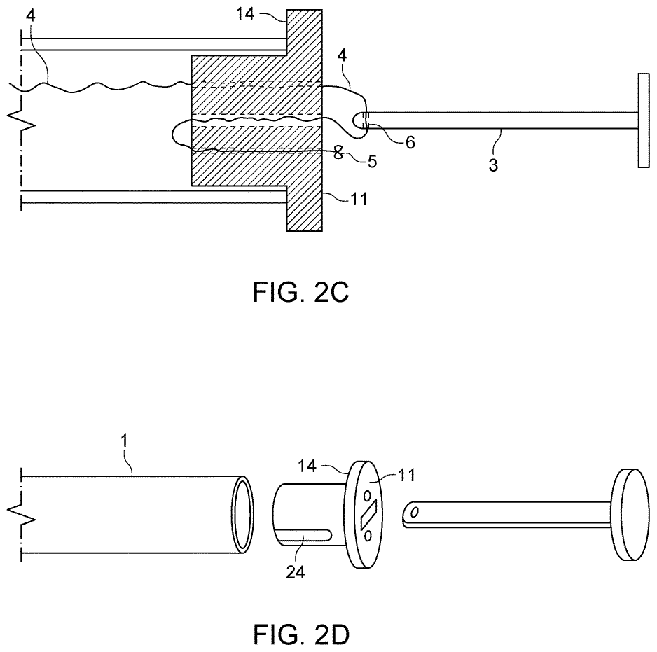

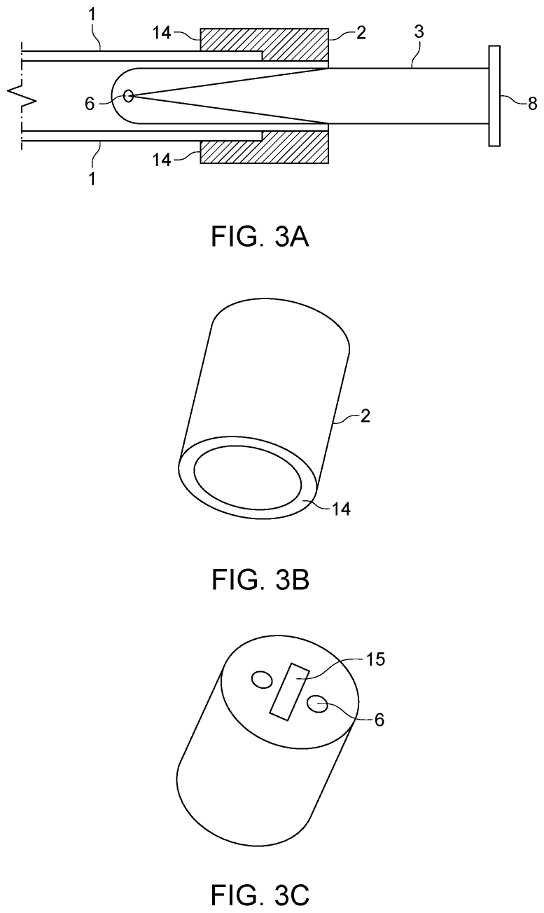

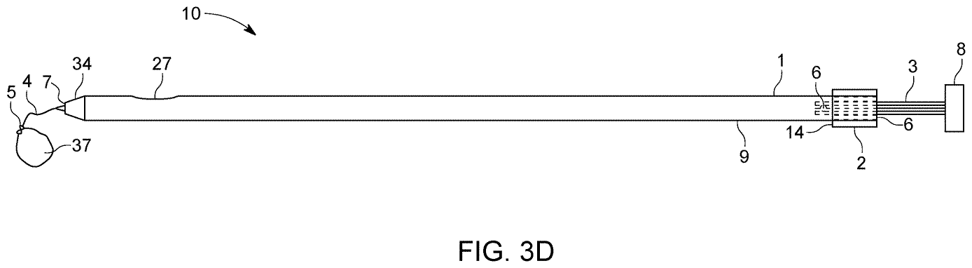

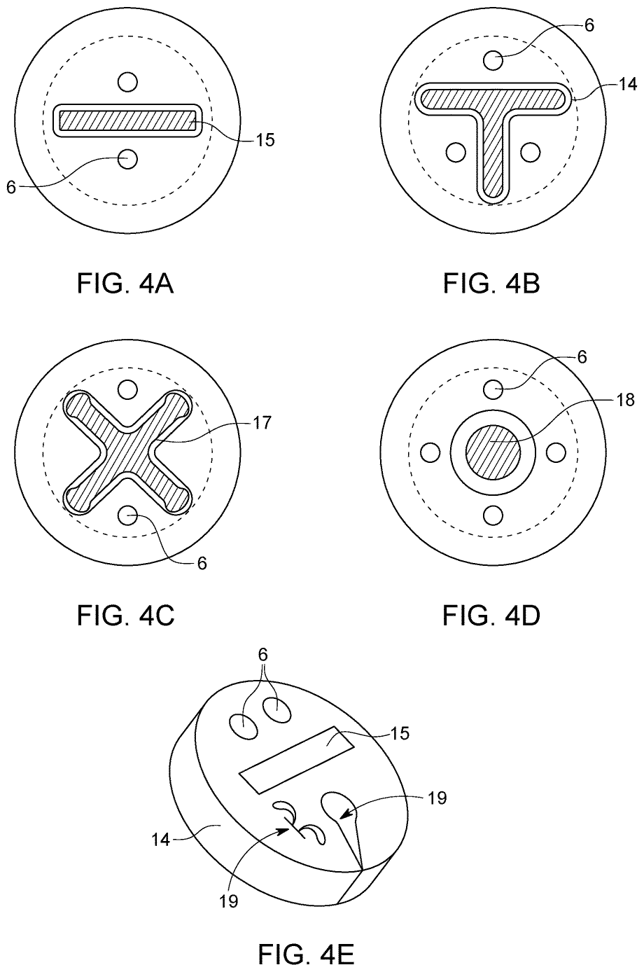

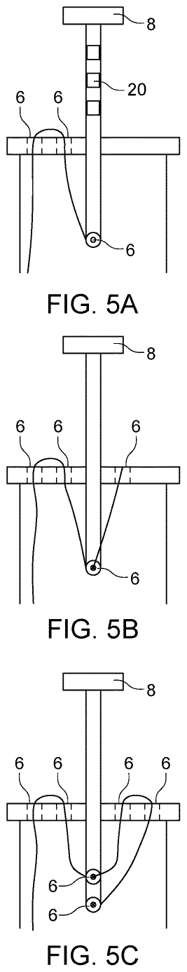

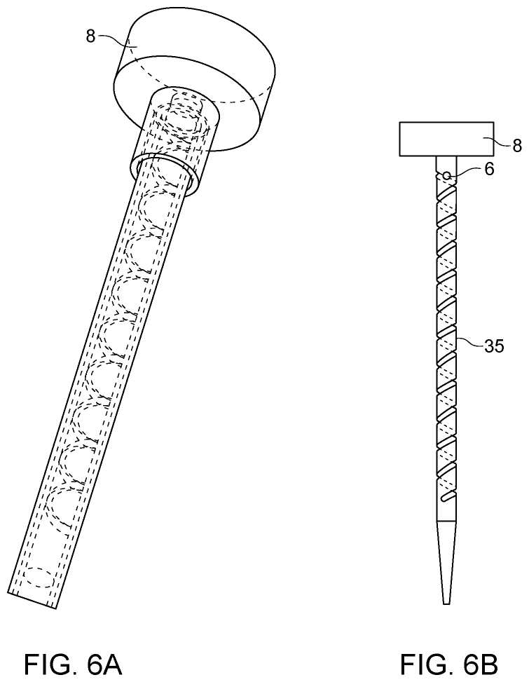

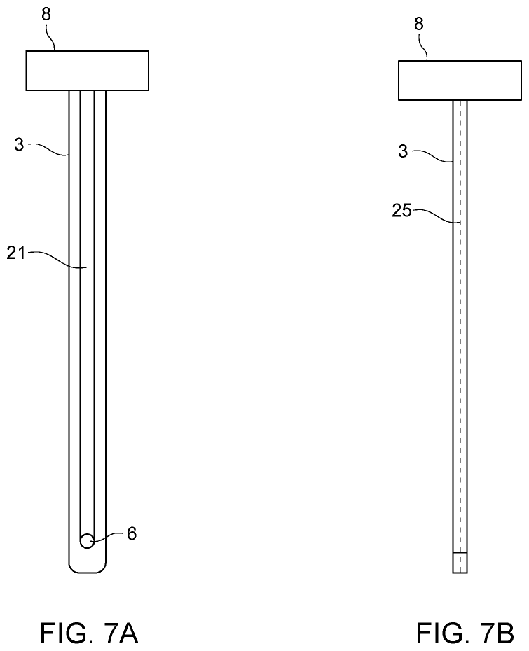

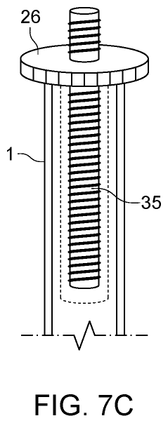

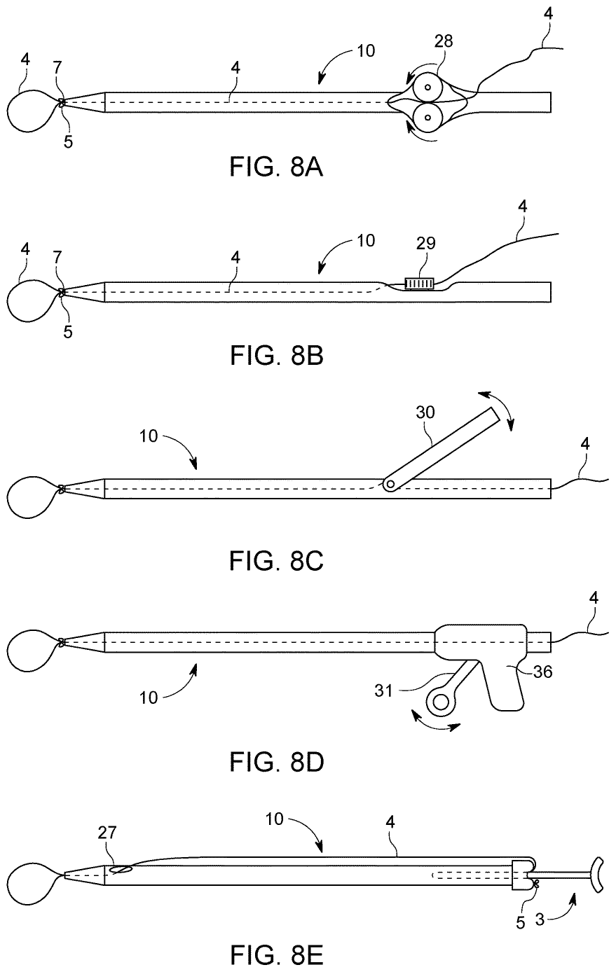

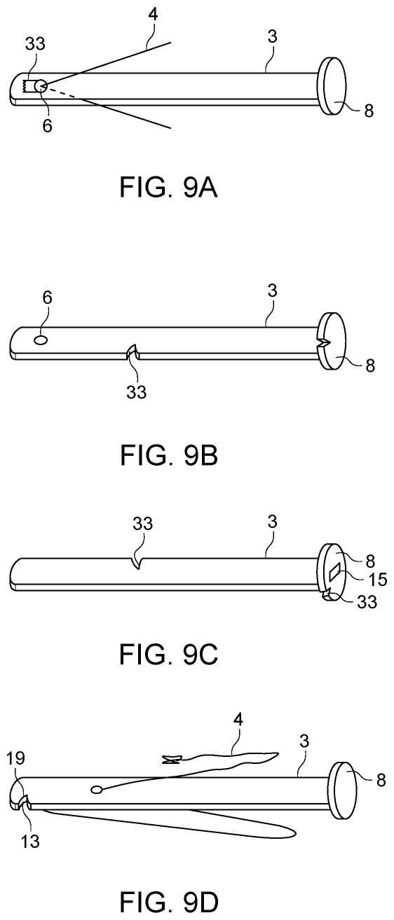

FIG. 2C is a perspective side view of an internal cap and an internal suture configuration of the single-handed ligation device according to an embodiment of the present invention.

FIG. 2D is an exploded view of the internal cap of the single-handed ligation device according to an embodiment of the present invention.

FIG. 3A is a perspective side view of the external cap and an internal suture configuration of the single-handed ligation device according to an embodiment.

FIG. 3B is a bottom and side perspective view of the external cap of the single-handed ligation device illustrating the cap's side and flange according to an embodiment.

FIG. 3C is a top and side perspective view of the external cap of the single-handed ligation device illustrating the cap's top and side, cap fulcrum holes, and the plunger slot according to an embodiment.

FIG. 3D illustrative view presenting a unibody configuration of a single-handed ligation device according to an embodiment of the present invention.

FIG. 4A is a top perspective view of an internal or external cap of the single-handed ligation device illustrating the cap's top, cap fulcrum holes, and a plunger slot according to an embodiment.

FIG. 4B is a top perspective view of an internal or external cap of the single-handed ligation device illustrating the cap's top, cap fulcrum holes, and a T shape plunger slot according to an embodiment.

FIG. 4C is a top perspective view of an internal or external cap of the single-handed ligation device illustrating the cap's top, cap fulcrum holes, and a cross shape plunger slot according to an embodiment.

FIG. 4D is a top perspective view of an internal or external cap of the single-handed ligation device illustrating the cap's top, cap fulcrum holes, and a round plunger slot according to an embodiment.

FIG. 4E is a top and side perspective view of an internal or external cap of the single-handed ligation device illustrating the cap's top and a side of the cap's flange, cap fulcrum holes, plunger slot, and a pinch cleat and/or tie cleat according to an embodiment.

FIG. 5A is a perspective view of the cap and plunger with finger holes illustrating a single suture leverage configuration of the single-handed ligation device according to an embodiment.

FIG. 5B is a perspective view of the cap and plunger illustrating a double suture leverage configuration of the single-handed ligation device according to an embodiment.

FIG. 5C is a perspective view of the cap and plunger illustrating a triple suture leverage configuration of the single-handed ligation device according to an embodiment

FIG. 6A is a perspective view of a rotating cap which provides suture leverage through radial motion of the cap relative to the single-handed ligation device according to an embodiment.

FIG. 6B is a perspective view of a for suture gathering through radial motion of the cap relative to the single-handed ligation device according to an embodiment.

FIG. 7A is a perspective view illustrating along one side of the plunger, a depressed track section of the plunger that provides a path for the suture to pass, and a plunger fulcrum hole of the single-handed ligation device according to an embodiment.

FIG. 7B is a perspective view illustrating along one side of the plunger of 7A. of the single-handed ligation device according to an embodiment.

FIG. 7C is a perspective view of a rotational cap that retract the suture and plunger from the single-handed ligation device according to an embodiment.

FIG. 8A is a perspective view of a finger wheel tensioner that utilizes the rotation to provide tension on and retraction of the suture via friction of the single-handed ligation device according to an embodiment.

FIG. 8B is a side perspective view of a finger wheel tensioner FIG. 8A of the single-handed ligation device according to an embodiment.

FIG. 8C is a perspective view of finger ratchet tensioner that utilizes repeated movement of the ratchet bar to generates tension on, and retraction of, the suture of the single-handed ligation device according to an embodiment.

FIG. 8D is a perspective view of a finger-trigger ratchet tensioner where repeated movement of the trigger element generates tension on and retraction of the suture of the single-handed ligation device according to an embodiment.

FIG. SE is a perspective view of an external suture configuration of the single-handed ligation device according to an embodiment.

FIG. 9A is a perspective view of a cutting device in the distal portion of the plunger fulcrum hole of the single-handed ligation device according to an embodiment.

FIG. 9B is a perspective view of a cutting device located on the plunger of the single-handed ligation device according to an embodiment.

FIG. 9C is a perspective view of a divot style cutting device of the knob of the plunger of the single-handed ligation device according to an embodiment.

FIG. 9D is a perspective view of pinch cleat on the distal end of the plunger of the single-handed ligation device according to an embodiment.

### DETAILED DESCRIPTION OF THE INVENTION

Terms or words used in this specification and claims should not be restrictively interpreted as ordinary meanings or dictionary-based meanings, but rather should be interpreted consistently with the concepts disclosed in the present application, based on the principle that an inventor can act as his/her own lexicographer, defining terms so as to best describe and explain his or her invention.

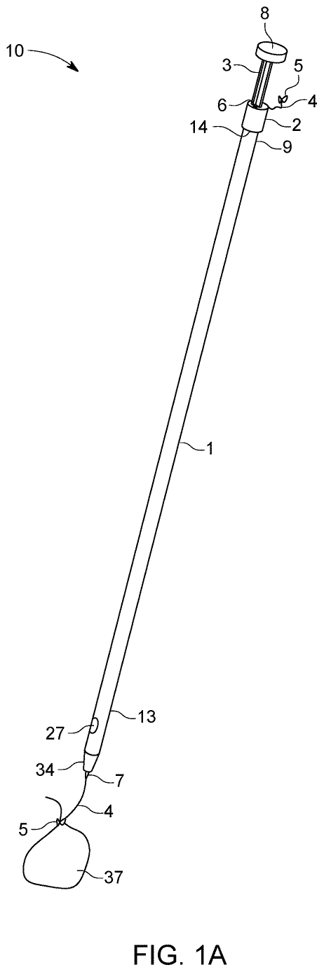

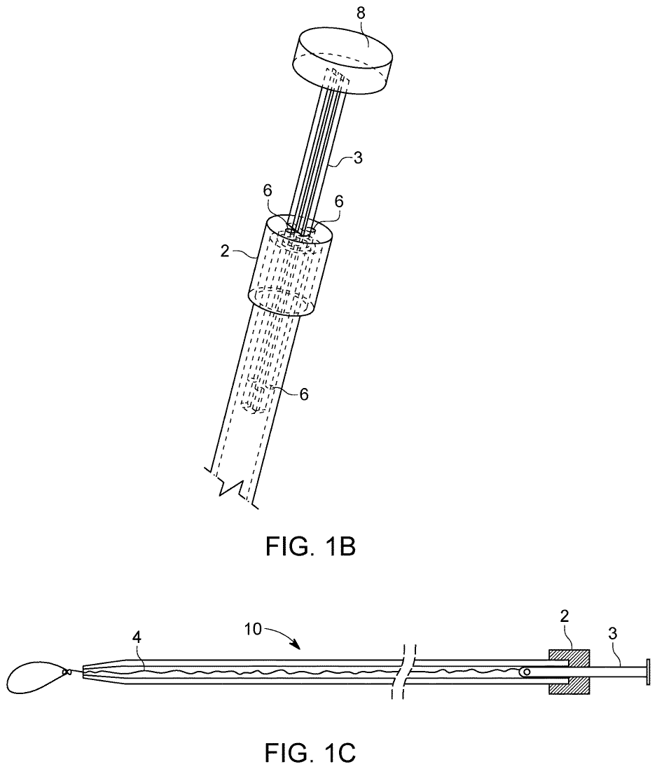

The single-handed ligation device (10) of the instant invention as presented in FIG. 1 can be used in laparoscopic procedures to ligate body tissues and the like. The device (10) operates easily with one hand and does not require the use of a second hand to retract suture (4) when ligating a structure. The device (10) employs, inter alia, a cannula (1) that performs as a rigid slender tool that can be used to apply pressure to and tighten a suture loop (37) having a pre-tied suture knot (5).

The single-handed ligation device (10) of FIG. 1 can be made of various materials, such as, different types of plastic. The use of plastic materials can make the single-handed ligation device (10) disposable. Alternatively, the single-handed ligation device (10) can be made of metal for purposes of sterilization and reuse.

Typically, the single-handed ligation device (10) is designed for use within a 5-millimeter Trocar. However, the cannula (1) can have any outer diameter equal to or less than the inside diameter of the Trocar. In FIG. 1A the cannula (1) has a tapered distal end (34) and a distal orifice (7) that is sized to let suture (4) pass smoothly through the orifice (7) when suture (4) is retracted internally into the cannula (1). The distal orifice (7) is sized (e.g., approximately 1-2 mm), so it does not permit the passage of the suture knot (5) into the device. According to an embodiment, FIG. 1B presents the proximal end (9) of the cannula (1), which can be designed and manufactured to accommodate various size caps (see e.g., FIG. 1B external cap (2), and e.g., FIG. 2B internal cap (11)) and plungers (3), as well as other various embodiments that include rotational round plungers (35), finger wheel tensioner (28), finger bar tensioner (29), finger rachet tensioner (30), finger-trigger rachet tensioner (31), or finger clutch tensioner (32), as needed (FIGS. 8A-8E) to provide rotation and/or tension on and retraction of the suture utilizing only one hand.

Not limited to any one size, the cannula (1) may be of any length required for use in different laparoscopic procedures (e.g., approximately 40-60 cm) and any length as needed with respect to the size of the patient. Further, the cannula (1) can incorporate, for example, a handle/trigger unit (36) (e.g., see FIG. 8D) at the proximal end to aid in grasping, as well as provide extra space for a plunger (3) and mechanical action.

Additionally, the cannula (1) can incorporate, for example, a “mechanical pencil” type means at the proximate end that provides a clutch tensioner that is not limited but can be a push type plunger (3), to retract the suture (4) inside (not shown) or outside of the cannula (1), see e.g., FIG. 8E.

According to an embodiment the single-handed ligation device (10) includes a flange at proximal end (9) to aid in plunging action, see e.g., FIGS. 2B-2D.

Typically, the internal and external caps (11and 2, respectively) include a flange (14). However, the flange (14) may be a part of or affixed to the cannula (1), irrespective of the cap (2, 11). The flange (14) design is not limited and may include any size or shape to aid in handling and/or gripping the device (10), so that the user can easily assert a downward force to quickly retract the suture (4) and tighten the suture knot (5). Specifically, the single-handed ligation device (10) is designed to be used with one hand, typically holding the flange with one's forefinger and middle finger, while asserting downward pressure on the knob (8) with one's thumb. The knob (8) is designed to provide comfort and stability and is not limited and may be shaped like a ring or a pad that provides secure surface contact when actuating the plunger (3).

The single-handed ligation device (10) can be “reloaded,” i.e., suture (4) replacement, for multiple deployments, as needed during a surgical procedure. Alternatively, the single-handed ligation device (10) can be manufactured for single disposable use. The device's (10) canula (1) can be hollow to retract suture (4) internal to the device (10), see e.g., FIG. 1A, or the canula (1) can be solid in part, wherein the suture (4) is retracted external to the device (10), see e.g., FIG. 8E.

The single handed ligation device (10) uses suture material to create the ligation. The suture (4) material can be made of compounds as known in the art, such as, nylon, polypropylene, silk, polyester, and the like. The suture (4) material is preferably stiff enough to form an open suture loop (37) for lassoing tissue, flexible enough to be knotable and tightened. It is helpful for the suture (4) material to have an extra stiffened proximal end to aid in threading the device (10) assembly. Preferably, the suture (4) material has an appropriate diameter to prevent excessive air/liquid loss through the distal end's (13) orifice (7). Importantly, the suture (4) material must be capable of accommodating a tightening suture knot (5). The suture (4) material also needs to be of sufficient length to accommodate a suture loop (37), knot (5), span the length of the cannula (1), pass though the cap (2, 11) and plunger (3), and if necessary to be tightened by the surgeon's hand grasp. The suture (4) material necessarily requires to be strong enough to pull against and tighten the suture knot (5) and can be permanent or dissolvable nature.

FIGS. 2A-2D, FIGS. 3A-3C, and FIGS. 4A-4E present various embodiments of the caps (2, 11) and their components of the single-handed ligation device (10).

The caps (2, 11) are attached to the proximal end (9) of the cannula (1) and provide control for the suture (4) and plunger (3). The caps (2, 11), which are not limited to any one design (e.g., they can be affixed to the top of the canula (1) or designed to affix externally/internally to the canula (1)), are attached to the proximal end (9) of the cannula (1). Additionally, the caps (2, 11) can be affixed permanently, or they can be removable. Further, caps (2, 11) can be external or internal rotating cap, e.g., FIG. 7C.

FIG. 3D presents an embodiment of the single-handed ligation device (10), wherein the cannula (1) and cap are manufactured to form one continuous piece to provide functionality, i.e., accommodation of the suture (4) and the plunger (3). According to this embodiment, the uniform cannula and cap provide a flange (14) or accommodation at the proximate end (9) to aid a user in the plunging action required to actuate the device (10). According to the embodiment the single-handed ligation device (10) having a uniform cannula and cap is preloaded with suture (4) a suture loop (37) having a pre-tied suture knot (5) during manufacturing, so that the plunger (3) and suture loop (37) are positioned for actuation and deployment. Accordingly, the single-handed ligation device (10) can be manufactured to be disposable after single use.

Alternatively, the internal and external caps (11, 2) of FIGS. 2B, 2C, 2D and FIG. 3A can be attached to the cannula (1) at the proximate end by known means, such as, mechanical action, compression fittings, screws, friction, threading, and the like, so that the caps (2, 11) can be easily removed to facilitate cleaning and/or reloading of suture (4), as needed. The caps (2, 11) can be made of any material including various plastics and/or metals and can surround the cannula (1) or extend inside of the cannula (1) to provide support to the device (10). The caps (2, 11) can be permanently affixed to the cannula (2) during manufacturing in the disposable version of the device (10).

Non-limiting design examples of the top surfaces of cap (2, 11) that may be incorporated into the device (10) are presented in FIGS. 4A-4E. FIGS. 4A-4E present various orifices, i.e., slot (15), e.g., T slot (16), cross slot (17), and round slot (18) for receiving a plunger (3) having these configurations. The various slot configurations (15, 16, 17, 18) are designed to provide control over the plunger (3), stability (under pressure), and speed of movement and may incorporate special material or devices to facilitate control of the plunger (3), as well as being able to accommodate the suture (4) material being pulled through the slot (15, 16, 17, 18) with the plunger (3). In addition, the plunger (3) may have at least one plunger track (21) to accommodate the smooth passage of suture (4).

Further, the cap (2, 11) contains a plurality fulcrum holes (6) that control, guide, and facilitate the movement of suture (4) during the device's (10) operation.

A plurality of cap (2, 11) fulcrum holes (6) may be described as a first cap, second cap, third cap etc. fulcrum hole (6) when describing the suture's (4) configured path through the device (10). It should be noted that the fulcrum holes (6) may be flared and/or smoothed or designed in any fashion, with any material that facilitates the suture's (4) ease of movement and loading. Additionally, the cap (2, 11) may provide a tether point utilizing, e.g., cleat (19) (not limited to any particular design) for affixing/anchoring the distal end of the suture (4).

According to an embodiment, FIGS. 2A and 2B present the proximate portion of the device (10), wherein suture (4) enters the distal orifice (7) (see FIG. 1) and immediately exits the cannula (1) through an exit orifice (27) (see FIG. 1) at the distal end of the cannula (1). From the exit orifice (27) the suture (4) remains external to the cannula (1) until reaches an indent (23) in the flange (14) of the internal cap (11) that provides a path for the suture (4) to cap fulcrum hole (6), where the suture (4) changes direction and proceeds to plunger fulcrum hole (6) at the distal end of the plunger (3), see FIG. 2B. In this regard, the device (10) may have an exit orifice (27) for suture (4) material to exit anywhere along the cannula (1), as needed. For example, an exit orifice (27) may be located at the tapered distal end (34) near the distal orifice (7), or at the proximal end (9) of the device (10). Additionally, one or more exit orifices (27) may be located in the internal or external cap (11, 2) depending on the suture (4) configuration.

The cap (2, 11) of the present invention has eased edges and/or specifically designed radii and/or surface profiles to allow smooth suture (4) movement with minimal friction. According to an embodiment of the invention, the cap (2, 11) can have a position key slot (24) (see FIG. 2D) with the cannula (1) to prevent rotation of the plunger (3) within the device (10). The cap (2, 11) and the plunger (3) may include a means, device or feature that allows for proximal end of the suture (4) to be cinched up and firmly attached via a knot or other mechanical gripping device such as a cam cleat (19) pinch cleat, jam cleat, cleat hinge or tapered pinching slot, and the like (see, e.g., FIG. 4E and FIG. 9D).

FIGS. 6A-6B and 7A-7C present non-limiting embodiments of the plunger (3) utilized by the single-handed ligation device (10) of the present invention. The plunger (3) provides mechanical actuation of suture (4). The plunger (3) is operated by hand and is specifically designed to integrate with the cap (2, 11) of the device (10). The plunger (3) length is not limited and can be e.g., approximately 12-16 cm in length and having a width that is ½ to ⅔ of the cannula's (1) inner diameter. The plunger (3) may have crenulations (25), or a tiny notch, or a scalloped edge along the shaft of the plunger (3) (see FIG. 7B), that work in connection with a counterpart (e.g., a crenulation means to facilitate movement) within slot (15) to steady and/or assist in the actuation of the plunger 3). The plunger (3) can be made of various materials, e.g., plastic or metal, and provides bending strength to promote only longitudinal movement under compression and in tension. In FIG. 6A external cap (2) comprises a knob (8), e.g., a round pad at the proximal end for applying force with finger or thumb that is affixed to plunger (3), which can be rotated.

The plunger (3) may have intermediate pads, edges, crenulations or finger holes (20) along the length of the plunger (3) to allow multiple stage pushes and facilitate longer travel lengths, see e.g., FIG. 5A. Alternatively, the plunger (3) can be round or any variety of cross sections, see e.g., FIG. 6B and FIG. 7C. The round plunger (3) (see FIG. 6B) can provide an area or areas where the suture (4) can attach or pass through, for example, plunger fulcrum hole (6) and the round plunger (3) functions in a similar manner to the above-described additional plunger shapes. Alternately the round plunger (3) depicted in FIG. 6A and FIG. 6B do not move in and out of the cannula (2) and instead are rotated to retract the suture (4) in a spool like fashion.

The plunger (3) can be keyed with the cap (2, 11) via slot (15), to limit rotation within the assembly. The plunger (3) is sized to fit within an enlarged proximate end (9) region of the cannula (1) or simply within the cannula's (1) inner diameter. Moreover, the size of the plunger (3) approximates the inside diameter of the cannula (2), to not allow the plunger to deflect and/or distort as it is actuated. The inner diameter of the cannula (1) or proximate end (9) region can provide the longitudinal support needed to prevent the plunger (3) from bending. The plunger (3) is designed to provide distinct areas for suture (4) and or other apparatuses to pass through the slot (15).

The plunger (3) may have a plurality of fulcrum holes (6) at its distal end, and they are described as a plunger first, second, third, etc. fulcrum hole (6) to describe the suture's (4) configured path through the device (10) and where the proximal end of the suture (4) may be tethered to prevent movement. The plunger fulcrum hole (6) can be used to anchor the suture (4). The suture (4) can be tied to the plunger fulcrum hole (6) or secured by tying the suture's (4) proximate end in a knot that cannot pass through the fulcrum hole (6). Alternatively, the distal end of the plunger (3) may provide a tether point utilizing, e.g., cleat (19) for affixing/anchoring the distal end of the suture (4), see e.g., FIG. 9D. Further, the plunger fulcrum hole (6) in the distal end of the plunger can provide a fulcrum or pivot point for the suture (4) to pass through and change direction within the device (10). Furthermore, the plunger (3) may have eased edges and/or specifically designed radii and or surface profiles to provide for smooth suture (4) movement with minimal friction.

The single-handed ligation device (10) conveniently provides increased mechanical leverage over the suture (4) material. Depending on how the suture's (4) path through the device (10) is configured the device (10) can provide, for example, a single suture leverage (or 1× suture leverage). A single suture leverage is a 1:1 ratio, i.e., the distance traveled by the plunger (3) from the initiation of plunger depression within the cap (2, 11) to the point where the plunger is fully depressed within the device (10) is equal to the length of suture (4) retracted into the cannula (1). A double suture leverage (or 2× suture leverage) is a 1:2 ratio, i.e., the distance traveled by the plunger (3) from the initiation of plunger depression within the cap (2, 11) to the point where the plunger is fully depressed within the device (10) provides for the length of suture (4) retracted into the cannula (1) to be twice the length of the distance traveled by the plunger (3). A triple suture leverage (or 3× suture leverage) is a 1:3 ratio, i.e., the distance traveled by the plunger (3) from the initiation of plunger depression within the cap (2, 11) to the point where the plunger is fully depressed within the device (10) provides for the length of suture (4) retracted into the cannula (1) to be three times the length of the distance traveled by the plunger (3). The suture (4) of the single-handed ligation device (10) can be configured to provide at least a 1× suture leverage, and more preferably multiple suture leverages, such as 2× suture leverage, 3× suture leverage, or more. Thus, the rate at which the suture (4) is retracted into the distal orifice (7) and how quickly the suture loop (37) can be closed is directly dependent on the configuration of the suture (4) in and around the device (10).

In this regard, FIG. 5A presents an embodiment of the invention where the device (10) provides a single (1×) suture leverage. FIG. 5A illustrates this with the plunger (3) midway through actuation and suture (4) ascending from the distal end (13) of the cannula (1) and the proximate end of the suture (4) passing through a first cap fulcrum hole (6) and reversing direction down a second cap fulcrum hole (6) (alternatively, reversing direction down slot (15), i.e., adjacent to the plunger (3)), where the proximate end of suture (4) is tied securely to a first plunger fulcrum hole (6) at the distal end of the plunger (3). According to this suture (4) configuration, when a user depresses the plunger (3) the suture (4) is retracted by the cannula (1) a distance that is equal to the distance that the plunger (3) travel after being fully depressed. As such, this embodiment provides a single or 1× suture leverage, i.e., the distance ratio is 1:1 and the distance the plugger travels is equal to the length of suture (4) that is retracted.

FIG. 5B presents an embodiment of the invention where the device (10) provides a double or 2× suture leverage. FIG. 5B illustrates this with the plunger (3) midway through actuation and the suture (4) ascends from the distal end (13) of the cannula (1) and the proximate end of the suture (4) passing through a first cap fulcrum hole (6) reversing direction down a second cap fulcrum hole (6) (alternatively, reversing direction down slot (15)), and then reversing course passing through a first plunger fulcrum hole (6) at the distal end of the plunger (3), and then the proximate end of suture (4) is tethered (e.g., by suture knot, cleat (19) not shown) to a third cap fulcrum hole (6). According to this suture (4) configuration, when a user depresses the plunger (3) the suture (4) is retracted by the cannula (1) a distance that is equal to double (or 2×) the distance that the plunger (3) travels after being fully depressed. As such, this embodiment provides a double or 2× suture leverage, i.e., the distance ratio is 1:2 and the distance that the plunger traveled is one-half the length of suture (4) that is retracted.

FIG. 5C presents an embodiment of the invention where the device (10) provides a double or 3× suture leverage. FIG. 5C illustrates this with plunger (3) midway through actuation and the suture (4) ascends from the distal end (13) of the cannula (1) and the proximate end of the suture (4) passing through a first cap fulcrum hole (6) reversing direction down a second cap fulcrum hole (6) (alternatively, reversing direction down slot (15)), and then reversing direction passing through a first plunger fulcrum hole (6) at the distal end of the plunger (3), and passing through a third cap fulcrum hole (6) reversing direction down a fourth cap fulcrum hole where the proximate end of suture (4) is tied securely to a second plunger fulcrum hole (6) at the distal end of the plunger (3). According to this suture configuration, when a user depresses the plunger (3) the suture (4) is retracted by the cannula (1) a distance that is equal to triple (or 3×) the distance that the plunger (3) travels after being fully depressed. As such, this embodiment provides a triple or 3× suture leverage, i.e., the distance ratio is 1:3 and the distance that the plunger (3) traveled is one-third the length of the suture (4) that is retracted.

The suture leverage efficiencies, i.e., ratios of the distance traveled by the plunger (3) to suture (4) length retracted (1:1, 1:2, 1:3, or any ratio as needed), can be increased by increasing the number of fulcrum holes (6) in the cap (2, 11) and plunger (3) and maintaining that the proximate end of the suture (4) is anchored or tethered to either the cap (2, 11) or distal end of the plunger (3).

It should be noted that the plunger (3) of the device (10) can be removed after the suture knot (5) has been deployed and that the plunger (3) provides access to the proximal suture (4) end for suture (4) cutting outside of the patient. Further, the suture (4) can be loosened from the plunger (3) and/or cap (2, 11) to allow cutting of suture within the patient.

The embodiments of the invention as presented in FIGS. 9A-9D, optionally provide the single-handed ligation device (10) with a cutting device (33) or apparatus. The cutting device (33) for cutting the suture after deployment is designed to be operational with one-hand. The cutting device (33) can be in various areas of the singled-handed ligation device (10), for example, it can be located so that the suture (4) can be close to the tightened suture knot (5) or outside of the Trocar port and patient. The cutting device (33) can be integrated as part of the cannula (1), cap (2,11) or plunger (3). The cutting device (33) can include an actuating rod which pushes the cutting head into and against the tapered section of the cannula (1) triggering a blade to cut the suture (4). The blade of the cutting device (33) can be one or multiple pieces and the blade could be made of metal or other material. In this regard, the entire cutting device (33) can be made of metal or other materials. The cutting device (33) can work in concert with the tensioning of the suture (4) as provided by the plunger (3). The tapered distal end (34) of the cannula (1) actuates the depression of the blade. The cutting device (33) may be a deforming piece of plastic that when compressed exposes small blade. The blade of the cutting device (4) may be a one-use blade that once deployed, does not retract, and is discarded and replaced. If the cutting device is integrated within the cannula (1), it must be positioned to allow the suture (4) to pass freely for loading and actuating of the device (10).

Further embodiments of the single-handed ligation device (10) are contemplated and are directed to a hinged cannula (1) that when moved provides tightening of the suture (4). Other embodiments include a spring-loaded plunger (3) that can be used with collets for grabbing and releasing the suture (4), wherein the plunger (3) can be pumped like, e.g., a mechanical pencil to advance the suture (4) in either a single clutch or double clutch design. Additional, embodiments of the single-handed ligation device (10) included a hinged ratcheting mechanism, knurled wheel which grabs and pulls on the suture (4).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The entire disclosure of each of the patent documents and scientific articles referred to herein is incorporated by reference for all purposes.

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. The scope of the disclosure is thus indicated by the appended claims rather than by the foregoing description, and all changes that come within the meaning and range of equivalency of the claims are intended to be embraced therein.

What is claimed is:

1. A single-handed ligation device for tying a suture around a selected body tissue, said device comprising: a suture having a proximate end and a distal end containing a suture loop and a suture knot external to the device, an elongated cannula extending axially between a proximate end and a distal end, the distal end having a tapered end containing a distal orifice sized to provide passage for only the suture, a cap having an open end fixedly attached to the proximate end of the cannula and a closed end containing a slot and at least a first cap fulcrum hole and a flange encircling the cannula, a plunger having a distal end opposite a proximate end having a knob, the distal end being sized for axial movement through the slot and proximate end of the cannula, the plunger having at least a first plunger fulcrum hole at the distal end, wherein the suture can be configured to provide a plurality increasing suture leverages.

2. The single-handed ligation device of claim 1, wherein the device provides a single suture leverage when the plunger is depressed, and suture is configured to ascend from distal end of the cannula where the proximate end of the suture passes through a first cap fulcrum hole and then reverses direction down a second cap fulcrum hole or the slot where the proximate end of suture is then tethered to a first plunger fulcrum hole at the distal end of the plunger.

3. The single-handed ligation device of claim 1, wherein the device provides a double suture leverage when the plunger is depressed, and suture is configured to ascend from distal end of the cannula where the proximate end of the suture passes through a first cap fulcrum hole and then reverses direction down a second cap fulcrum hole or the slot and further reverses direction passing through a first plunger fulcrum hole at the distal end of the plunger where the proximate end of suture is then tethered to a third cap fulcrum hole.

4. The single-handed ligation device of claim 1, wherein the device provides a triple suture leverage when the plunger is depressed, and suture is configured to ascend from distal end of the cannula where the proximate end of the suture passes through a first cap fulcrum hole and then reverses direction down a second cap fulcrum hole or the slot and further reverses direction passing through a first plunger fulcrum hole at the distal end of the plunger and then the proximate end of suture passes through a third cap fulcrum hole and further reverses direction down a fourth cap fulcrum hole where the proximate end of suture is then tethered to a second plunger fulcrum hole.

5. The single-handed ligation device of claim 2, wherein the single suture leverage configuration provides a 1:1 ratio of the distance traveled by the plunger is equal to the length of suture retracted by the device.

6. The single-handed ligation device of claim 3, wherein the double suture leverage configuration provides a 1:2 ratio of the distance traveled by the plunger is equal to one-half the length of suture retracted by the device.

7. The single-handed ligation device of claim 4, wherein the triple suture leverage configuration provides a 1:3 ratio of the distance traveled by the plunger is equal to one-third the length of suture retracted by the device.

8. The single-handed ligation device of claim 1, wherein the cap has a T shaped slot, or a cross shaped slot, or round shaped slot and the plunger's distal end is appropriately configured and sized for axial movement through the slot and proximate end of the cannula.

9. The single-handed ligation device of claim 1, wherein the cap and cannula are manufactured as one piece.

10. The single-handed ligation device of claim 1, wherein the device contains a cutting device for cutting the suture after deployment.

11. The single-handed ligation device of claim 10, wherein the cutting device for cutting the suture is positioned on the distal end of the plunger and/or integrated as part of the cannula.

12. The single-handed ligation device of claim 2, wherein the proximate end of the suture after passing through a first cap fulcrum hole reverses direction down through the slot.

13. The single-handed ligation device of claim 3, wherein the proximate end of the suture after passing through a first plunger fulcrum reverses direction up through the slot.

14. The single-handed ligation device of claim 2, wherein the proximate end of suture is tethered to a cleat on the distal end of the plunger.

15. The single-handed ligation device of claim 1, wherein the device is made of a plastic material and is disposable.

16. The single-handed ligation device of claim 1, wherein the device is made of a metal material and can be sterilized.

17. The single-handed ligation device of claim 1, wherein the plunger can be removed after the suture knot has been deployed.

18. The single-handed ligation device of claim 1, wherein the proximate end of the cannula accommodates a means for retraction of the suture into the device.

19. The single-handed ligation device of claim 1, wherein the proximate end of the cannula accommodates a rotational round plunger, or a finger wheel tensioner, or a finger bar tensioner, or a finger rachet tensioner, or a finger-trigger rachet tensioner, or finger clutch tensioner, for retraction of the suture into the device.

20. The single-handed ligation device of claim 1, wherein the cannula is a hinged cannula that when moved provides retraction of the suture into the device, and/or wherein the device further comprises a spring-loaded plunger comprising collets for grabbing and releasing the suture.
